(19) United States
(12) Patent Application Publication
(10) Pub. No.: US 2023/0405279 A1
(43) Pub. Date: Dec. 21, 2023

(54) GUIDEWIRE POSITIONING DEVICES AND SYSTEMS

(71) Applicant: EDWARDS LIFESCIENCES CORPORATION, Irvine, CA (US)

(72) Inventors: Bingquan Su, Irvine, CA (US); Nima V. Nia, Mission Viejo, CA (US); Douglas Thomas Dominick, Irvine, CA (US); Sakyasingh Tripathy, San Diego, CA (US); Hengchu Cao, Irvine, CA (US); Rene Santana, Santa Ana, CA (US)

(21) Appl. No.: 18/239,696

(22) Filed: Aug. 29, 2023

Related U.S. Application Data
(63) Continuation of application No. PCT/US2022/017028, filed on Feb. 18, 2022.
(60) Provisional application No. 63/158,237, filed on Mar. 8, 2021.

(51) Int. Cl. A61M 25/04

(52) U.S. Cl. CPC A61M 25/04; A61M 2210/125

(57) ABSTRACT

A system for delivering a guidewire through a heart valve comprises a delivery catheter configured to pass through a blood flow pathway of a heart, an inner catheter having an inner lumen, a guidewire configured to pass through the inner lumen of the inner catheter, and a positioning device crimped onto an outer surface of the inner catheter. wherein. The delivery catheter is configured to receive the inner catheter, guidewire, and positioning device and is configured to prevent expansion of the positioning device. The positioning device is configured to expand upon removal from the delivery catheter.

### RELATED APPLICATION

This application is a continuation of International Patent Application No. PCT/US2022/017028, filed Feb. 18, 2022, and entitled GUIDEWIRE POSITIONING DEVICES AND SYSTEMS, which claims priority to U.S. Provisional Application No. 63/158,237, filed on Mar. 8, 2021, entitled GUIDEWIRE POSITIONING DEVICES AND SYSTEMS, the disclosure of which is hereby incorporated by reference in its entirety.

### BACKGROUND

### Field

The present disclosure generally relates to the field of heart valve correction.

### Description of Related Art

Heart valves can become calcified over time, which may prevent the heart valves from opening fully. As a result, calcified heart valves may provide relatively narrow openings for delivery of medical instruments.

### SUMMARY

For purposes of summarizing the disclosure, certain aspects, advantages and novel features have been described herein. It is to be understood that not necessarily all such advantages may be achieved in accordance with any particular embodiment. Thus, the disclosed embodiments may be carried out in a manner that achieves or optimizes one advantage or group of advantages as taught herein without necessarily achieving other advantages as may be taught or suggested herein.

Some implementations of the present disclosure relate to a system for delivering a guidewire through a heart valve. The system comprises a delivery catheter configured to pass through a blood flow pathway of a heart, an inner catheter having an inner lumen, a guidewire configured to pass through the inner lumen of the inner catheter, and a positioning device crimped onto an outer surface of the inner catheter. The delivery catheter is configured to receive the inner catheter, guidewire, and positioning device, the delivery catheter is configured to prevent expansion of the positioning device, and the positioning device is configured to expand upon removal from the delivery catheter.

The positioning device may be configured to conform to the blood flow pathway upon removal from the delivery catheter. In some embodiments, the positioning device is configured to, upon removal from the delivery catheter, position the inner catheter and guidewire above a central portion of the heart valve.

In some embodiments, the positioning device is at least partially composed of braided cords. The positioning device may have a generally cylindrical shape with generally flat sides configured to extend along the blood flow pathway to prevent tilting of the positioning device.

The positioning device may comprise a base portion, one or more side portions extending from the base portion and around a hollow interior of the positioning device, and an opening across from the base portion and configured to be positioned near the heart valve to collect fragments displaced from the heart valve.

In some embodiments, the positioning device is configured to expand to a diameter that is less than a diameter of the blood flow pathway to allow the positioning device to move along the blood flow pathway after the positioning device expands.

The positioning device may comprise one or more cords coiled about the inner catheter. In some embodiments, the one or more cords are configured to increase in diameter at a base portion of the positioning device and maintain a maximal diameter at a side portion of the positioning device.

In some embodiments, the positioning device comprises one or more flexible cords configured to curve away from the inner catheter and extend along the blood flow pathway. The positioning device may be configured to attach at multiple portions of the outer surface of the inner catheter.

In accordance with some implementations of the present disclosure, a method for delivering a guidewire through a heart valve comprises delivering a delivery catheter via a blood flow pathway to a heart valve. The delivery catheter has a first inner lumen configured to receive an inner catheter, a positioning device, and a guidewire. The method further comprises passing the inner catheter through the first inner lumen of the delivery catheter. The inner catheter has a second inner lumen and configured to carry the positioning device at an exterior surface of the inner catheter. The method further comprises extending the inner catheter out of a distal end of the delivery catheter to allow the positioning device to expand in multiple directions around the inner catheter and passing the guidewire through the second inner lumen of the inner catheter and out of a distal end of the inner catheter.

The positioning device may be configured to expand to position the inner catheter and guidewire above a central portion of the heart valve. In some embodiments, the positioning device has a generally cylindrical shape with generally flat sides configured to extend along the blood flow pathway to prevent tilting of the positioning device.

In some embodiments, the positioning device comprises a base portion, one or more side portions extending from the base portion and around a hollow interior of the positioning device, and an opening across from the base portion and configured to be positioned near the heart valve to collect fragments displaced from the heart valve.

The positioning device may comprise one or more cords coiled about the inner catheter. In some embodiments, the positioning device comprises one or more flexible cords configured to curve away from the inner catheter and extend along the blood flow pathway.

Some implementations of the present disclosure relate to a device for positioning a guidewire at an opening of a heart valve. The device comprises a crimped portion configured to crimp to an inner catheter, an expandable base portion configured to compress to fit within a delivery catheter and expand within a blood flow pathway upon removal from the delivery catheter, and one or more side portions configured to extend along tissue walls of the blood flow pathway to prevent tilting of the inner catheter.

In some embodiments, the expandable base portion is at least partially composed of braided cords. The expandable base portion may comprise one or more cords coiled about the inner catheter.

The expandable base portion and one or more side portions may comprise one or more flexible cords configured to curve away from the inner catheter and extend along the blood flow pathway.

### BRIEF DESCRIPTION OF THE DRAWINGS

Various embodiments are depicted in the accompanying drawings for illustrative purposes, and should in no way be interpreted as limiting the scope of the inventions. In addition, various features of different disclosed embodiments can be combined to form additional embodiments, which are part of this disclosure. Throughout the drawings, reference numbers may be reused to indicate correspondence between reference elements.

FIG. 1 provides a cross-sectional view of a human heart.

FIG. 2 provides a cross-sectional view of the left ventricle and left atrium of an example heart.

FIG. 3 illustrates a healthy heart valve in an open state in accordance with one or more embodiments.

FIG. 4 illustrates an example heart valve having three leaflets in an open state while experiencing calcification in accordance with some embodiments described herein.

FIG. 5 illustrates an example heart valve having two leaflets in an open state while experiencing calcification in accordance with one or more embodiments described herein.

FIG. 6 illustrates a delivery process involving delivering a guidewire and/or similar device to a calcified valve of a heart, according to certain embodiments.

FIGS. 7A and 7B illustrate an example delivery system for delivering a positioning device and/or guidewire into a patient's body for treatment of a heart valve, in accordance with one or more embodiments of the present disclosure.

FIGS. 8A and 8B illustrate an example delivery system for delivering a positioning device and/or guidewire into a patient's body for treatment of a heart valve, in accordance with one or more embodiments of the present disclosure.

FIGS. 9A and 9B illustrate an example delivery system for delivering a positioning device and/or guidewire into a patient's body for treatment of a heart valve, in accordance with one or more embodiments of the present disclosure.

FIG. 10 illustrates an example delivery system for delivering a positioning device and/or guidewire into a patient's body for treatment of a heart valve, in accordance with one or more embodiments of the present disclosure.

FIGS. 11-1 and 11-2 are flowcharts illustrating steps of a process for delivering a guidewire and/or similar device through a valve which may be at least partially narrowed due to calcification and/or other factors, in accordance with one or more embodiments of the present disclosure.

FIGS. 12-1 and 12-2 provide images relating to steps of the process of FIGS. 11-1 and 11-2, in accordance with some embodiments.

### DETAILED DESCRIPTION

Although certain preferred embodiments and examples are disclosed below, inventive subject matter extends beyond the specifically disclosed embodiments to other alternative embodiments and/or uses and to modifications and equivalents thereof. Thus, the scope of the claims that may arise herefrom is not limited by any of the particular embodiments described below. For example, with respect to any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments; however, the order of description should not be construed to imply that these operations are order dependent. Additionally, the structures, systems, and/or apparatuses/devices described herein may be embodied as integrated components or as separate components. For purposes of comparing various embodiments, certain aspects and advantages of these embodiments are described.

The headings provided herein are for convenience only and do not necessarily affect the scope or meaning of the claimed invention.

Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein. Similar reference numbers may be used with respect to separate diagrams and/or embodiments; use of such similar, or identical, reference numbers should not be interpreted as necessarily identifying identical components, and may refer to separate features.

Heart valves can become calcified over time, which can prevent the heart valves from opening fully. As a result, calcified heart valves may provide relatively narrow openings for delivery of medical instruments. While calcification can occur at various heart valves, the aortic valve may be particularly susceptible to becoming calcified.

Calcification at the aortic valve can be problematic for various medical procedures, including transcatheter aortic valve replacement (TAVR) procedures. Many TAVR and/or other procedures may involve delivering one or more devices and/or systems through the aortic and/or other valve. For example, a guidewire and/or similar device may first be delivered through the aortic valve to allow additional devices to be delivered through the aortic valve along the guidewire. Devices delivered via a guidewire can include balloon-expandable and/or self-expandable devices, replacement valves, balloons, sheath, and/or other devices. In some cases, catheters may naturally press against side walls of the blood flow pathway during delivery. Thus, when a catheter arrives at a heart valve, the catheter may not generally be positioned above or below a central portion of the heart valve.

While some guidewires may have a generally curved structure and/or may be configured to be curved to navigate through various blood flow pathways and/or valves, it may be difficult in some cases for surgeons to control and/or twist a guidewire to navigate the guidewire through a heart valve. Moreover, delivery of the guidewire and/or other devices may be delivered in a direction opposite the natural blood flow through the valve. For example, a guidewire may be delivered from the aorta through the aortic valve and into the left ventricle, while natural blood flow may be from the left ventricle through the aortic valve and into the aorta. Moreover, blood flow through the value may generally be at an angle, which may cause devices above the valve to be pushed to the side of the blood flow pathway while an opening through the valve may be generally at and/or around a central portion of the valve.

Physicians may generally use fluoro imaging to gain access through the valve. However, fluoro imaging may be limited to two-dimensional imaging. With limited control of the guidewire and/or other devices, it can require hours for physicians to gain access through the valve. Moreover, the catheter, guidewire, and/or other devices can scrape against the valve and/or cause displacement of calcified fragments at the valve. Displaced calcified fragments can enter the blood stream, which can cause various complications.

These difficulties may be greatly increased in the case of calcified and/or stenotic valves. For example, calcification can cause a narrowing of the opening through the valve, thus requiring surgeons to be more precise in directing the guidewire through the valve. Moreover, as a valve opening becomes narrower due to calcification and/or stenosis, the blood flow velocity through the valve (which may generally be opposite the direction of delivery, for example in retrograde-crossing procedures) increases. For example, blood flow velocity through a healthy heart valve may generally be around 0.5 m/s. However, blood flow velocity through a stenotic heart valve can reach up to 5 m/s. This increase in blood flow velocity can further oppose and/or complicate delivery (e.g., retrograde-crossing) of the guidewire and/or similar devices through the valve, thus requiring even more time for delivery in some cases. Moreover, in some cases, a tip portion of a catheter delivery system may become caught between the heart valve and a tissue wall.

Some devices, systems, and/or methods described herein can provide improvements for retrograde-crossing and/or guiding guidewires and/or other devices through heart valves and particularly stenotic heart valves. In some embodiments, guidewires and/or delivery systems may be delivered via catheter (i.e., transcatheter) for various medical procedures. The term “catheter” is used herein according to its broad and ordinary meaning and may include any tube, sheath, steerable sheath, steerable catheter, and/or any other means for delivering medical instrumentation, for example via an inner lumen configured to receive and/or deliver medical instrumentation, such as for positioning within a blood flow pathway (e.g., the aorta). Various delivery systems may be configured to be shaped and/or compressed to fit into and/or around a catheter.

In some embodiments, a delivery system may comprise an inner catheter/sheath (which may include any means for supporting positioning devices, catheters, and/or similar devices) configured to provide support to a positioning device (which can include any means for positioning one or more catheters and/or guidewires). The inner catheter may have any size (e.g., approximately 5 French). The positioning device may have any of a variety of forms and/or may be configured to attach to the inner catheter (e.g., at an outer surface of the inner catheter). In some embodiments, the positioning device may be at least partially composed of a mesh and/or braided pattern of cords. The inner catheter and/or positioning device may be configured to fit within a catheter/sheath and/or the catheter/sheath may be configured to prevent expansion of the positioning device, to contain the positioning device, and/or to hold the positioning device on the inner catheter.

The positioning device may be configured to expand when removed and/or upon removal from the catheter/sheath. For example, a positioning device may be composed of braided, coiled, and/or bendable cords which may be configured to compress to fit within the catheter/sheath. Responsive to removal from the catheter/sheath, the various cords may be configured to naturally decompress and/or expand to form a larger profile (e.g., conform to a size of a blood vessel lumen in which the positioning device is situated). For example, the positioning device may be configured to naturally and/or manually conform to an inner lumen of a blood vessel. In some instances, the positioning device may be configured to provide structural support to the inner catheter while in an expanded form. The positioning device may be configured to facilitate retrograde-crossing of the heart valve by supporting the inner catheter and/or a guidewire delivered via the inner catheter.

The positioning device may be configured to be used similarly to a stent, in which the positioning device may be configured to anchor itself (e.g., through friction with the tissue walls and/or using anchoring elements) within a blood flow pathway. In some embodiments, the positioning device may be configured to expand to a size slightly larger than the blood flow pathway such that the positioning device may be configured to fit snugly within the blood flow pathway. However, it may not be necessary for the positioning device to fit snugly within the blood flow pathway. For example, the positioning device may have a diameter slightly less than a diameter of the blood flow pathway such that that the positioning device may form a loose anchor against the tissue walls of the blood flow pathway. The sides of the positioning device may be configured to bump into the walls of the blood flow pathway if the positioning device drifts a given distance from a center point of the blood flow pathway. In this way, the positioning device may be enabled to move along the blood flow pathway while in an expanded form within the blood flow pathway.

The inner catheter may be configured to pass at least partially through the positioning device and/or the positioning device may have a central connection to enable the positioning device to be physically attached to the inner catheter. In some embodiments, the inner catheter may be situated at a generally central position within the positioning device and/or the positioning device may be configured to position the inner catheter at a central position within a blood flow pathway and/or above or below a heart valve. A guidewire passed through the inner catheter may have a generally flexible structure that may make the guidewire susceptible to bending in response to blood flow through the heart valve. However, the inner catheter and/or positioning device may be at least partially rigid to direct the guidewire towards a desired point (e.g., a central portion) of the heart valve. The inner catheter and/or positioning device may be configured to be delivered adjacent to the heart valve such that the guidewire may be configured to enter the heart valve directly or near-directly upon exiting the inner catheter and/or positioning device.

One or more guidewires and/or similar devices may be configured to be passed through the inner catheter for delivery through the heart valve. The positioning device may be configured to position a guidewire such that the guidewire may be situated above or below a central point of the valve. Many heart valves, including stenotic heart valves, may be configured to open at least partially at a central point of the heart valves. The positioning device may be configured to prevent tilting of the inner catheter and/or guidewire such that when the guidewire exits the inner catheter, the guidewire may be pointed generally towards a central point of the heart valve. Thus, delivery of the guidewire through the valve may be simplified for physicians and/or less time may be required for delivery.

In some embodiments, the positioning device may be configured to catch and/or collect calcified fragments displaced from the heart valve by the guidewire, inner sheath, positioning device, and/or other delivery systems. For example, the positioning device may have a basket- and/or bowl-shape form with a hollow interior to enable collection of calcified fragments at or near the positioning device. In this way, calcified fragments may advantageously be prevented from further progressing in the blood stream and/or causing various complications. The positioning device may be collapsed and/or compressed to retract into a delivery catheter. Some calcified fragments within the positioning device may be retained within the positioning device when the positioning device is collapsed/compressed.

The positioning device may be configured to be removed following delivery of a guidewire through the heart valve or may be retained to facilitate delivery of other devices through and/or at the heart valve. For example, the positioning device may be configured to facilitate delivery of a replacement heart valve to prevent the replacement heart valve from being misplaced (e.g., placed at an angle).

In some embodiments, a delivery catheter used for delivering a guidewire, positioning device, and/or other delivery systems may be configured for delivering treatment devices (e.g., TAVR devices, replacement valves, etc.). For example, a TAVR device may be crimped onto an outer surface of a delivery catheter configured to deliver the guidewire and/or positioning device. Thus, the delivery catheter may advantageously not be required to be removed from a patient's body following delivery of the guidewire and/or additional devices for delivering the treatment devices may not be required.

Example positioning devices described herein may be composed of one or more of a variety of materials. For example, a positioning device may be at least partially composed of Nitinol and/or other shape-memory alloys and/or plastic (e.g., bioresorbable plastics).

In humans and other vertebrate animals, the heart generally comprises a muscular organ having four pumping chambers, wherein the flow thereof is at least partially controlled by various heart valves, namely, the aortic, mitral (or bicuspid), tricuspid, and pulmonary valves. The valves may be configured to open and close in response to a pressure gradient present during various stages of the cardiac cycle (e.g., relaxation and contraction) to at least partially control the flow of blood to a respective region of the heart and/or to blood vessels (e.g., pulmonary, aorta, etc.).

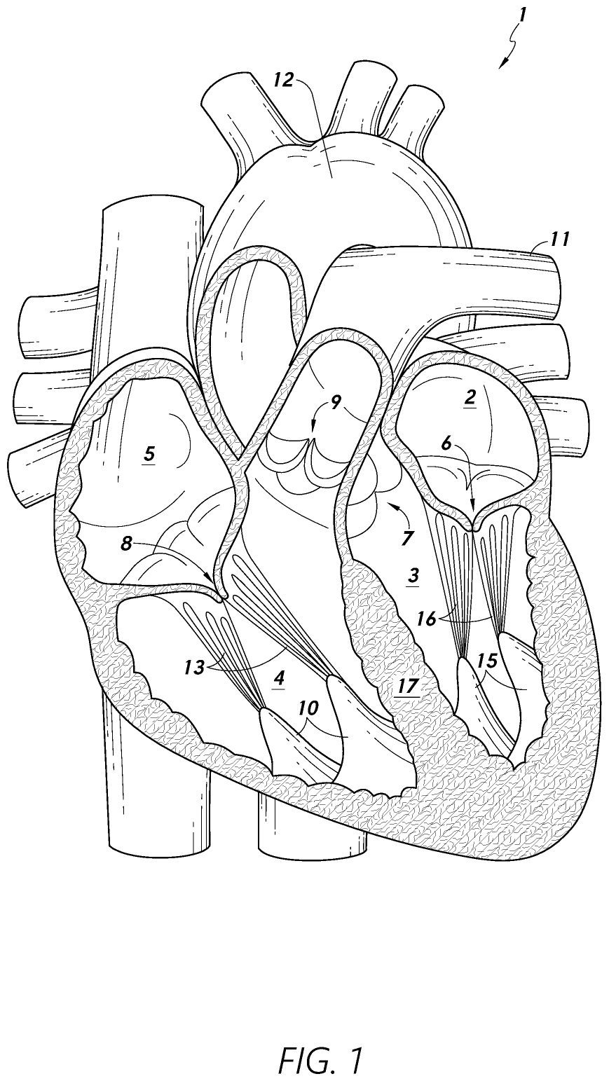

FIG. 1 illustrates an example representation of a heart 1 having various features relevant to certain embodiments of the present inventive disclosure. The heart 1 includes four chambers, namely the left atrium 2, the left ventricle 3, the right ventricle 4, and the right atrium 5. A wall of muscle 17, referred to as the septum, separates the left 2 and right 5 atria and the left 3 and right 4 ventricles. The heart 1 further includes four valves for aiding the circulation of blood therein, including the tricuspid valve 8, which separates the right atrium 5 from the right ventricle 4. The tricuspid valve 8 may generally have three cusps or leaflets and may generally close during ventricular contraction (i.e., systole) and open during ventricular expansion (i.e., diastole). The valves of the heart 1 further include the pulmonary valve 9, which separates the right ventricle 4 from the pulmonary artery 11, and may be configured to open during systole so that blood may be pumped toward the lungs, and close during diastole to prevent blood from leaking back into the heart from the pulmonary artery. The pulmonary valve 9 generally has three cusps/leaflets, wherein each one may have a crescent-type shape. The heart 1 further includes the mitral valve 6, which generally has two cusps/leaflets and separates the left atrium 2 from the left ventricle 3. The mitral valve 6 may generally be configured to open during diastole so that blood in the left atrium 2 can flow into the left ventricle 3, and advantageously close during diastole to prevent blood from leaking back into the left atrium 2. The aortic valve 7 separates the left ventricle 3 from the aorta 12. The aortic valve 7 is configured to open during systole to allow blood leaving the left ventricle 3 to enter the aorta 12, and close during diastole to prevent blood from leaking back into the left ventricle 3.

Heart valves may generally comprise a relatively dense fibrous ring, referred to herein as the annulus, as well as a plurality of leaflets or cusps attached to the annulus. Generally, the size and position of the leaflets or cusps may be such that when the heart contracts, the resulting increased blood pressure produced within the corresponding heart chamber forces the leaflets at least partially open to allow flow from the heart chamber. As the pressure in the heart chamber subsides, the pressure in the subsequent chamber or blood vessel may become dominant, and press back against the leaflets. As a result, the leaflets/cusps come in apposition to each other such that the leaflets/cusps coapt, thereby closing the flow passage.

The atrioventricular (i.e., mitral 6 and tricuspid 8) heart valves may further comprise a respective collection of chordae tendineae (16, 13) and papillary muscles (15, 10) for securing the leaflets of the respective valves to promote and/or facilitate proper coaptation of the valve leaflets and prevent prolapse thereof. The papillary muscles (15, 10) may generally comprise finger-like projections from the ventricle wall, while the chordae tendineae (16, 10) may comprise cord-like tendons that connect the papillary muscles to the valve leaflets.

With respect to the mitral valve 6, a normal mitral valve may comprise two leaflets (anterior and posterior) and chordae tendineae 16 connecting the leaflets to two corresponding papillary muscles 15. The papillary muscles 15 originate in the left ventricle wall and project into the left ventricle 3. The valve leaflets of the mitral valve 6 may be prevented from prolapsing into the left atrium 2 by the action of the chordae tendineae 16 tendons connecting the valve leaflets to the papillary muscles 15. The relatively inelastic chordae tendineae 16 are attached at one end to the papillary muscles 15 and at the other to the valve leaflets; chordae tendineae from each of the papillary muscles 15 are attached to a respective leaflet of the mitral valve 6. Thus, when the left ventricle 3 contracts, the intraventricular pressure can force the valve to close, while the chordae tendineae 16 may keep the leaflets coapting together and prevent the valve from opening in the wrong direction, thereby preventing blood to flow back to the left atrium 2. The various cords of the chordae tendineae may have different thicknesses, wherein relatively thinner cords are attached to the free leaflet margin, while relatively thicker cords (e.g., strut cords) are attached farther away from the free margin.

With respect to the tricuspid valve 8, a normal tricuspid valve may comprise three leaflets (two shown in FIG. 1) and three corresponding papillary muscles 10 (two shown in FIG. 1). The leaflets of the tricuspid valve 8 may be referred to as the anterior, posterior and septal leaflets, respectively. The valve leaflets are connected to the papillary muscles by the chordae tendineae 13, which are disposed in the right ventricle 4 along with the papillary muscles 10. Although tricuspid valves are described herein as comprising three leaflets, it should be understood that tricuspid valves may occur with two or four leaflets in certain patients and/or conditions; the principles relating to papillary muscle binding and/or adjustment disclosed herein are applicable to atrioventricular valves having any number of leaflets and/or chordae tendineae or papillary muscles associated therewith. The right ventricular papillary muscles 10 originate in the right ventricle wall, and attach to the anterior, posterior and septal leaflets of the tricuspid valve, respectively, via the chordae tendineae 13. The papillary muscles 10 may serve to secure the leaflets of the tricuspid valve 8 to prevent prolapsing of the leaflets into the right atrium 5 during ventricular systole. Tricuspid regurgitation can be the result of papillary dysfunction or chordae rupture.

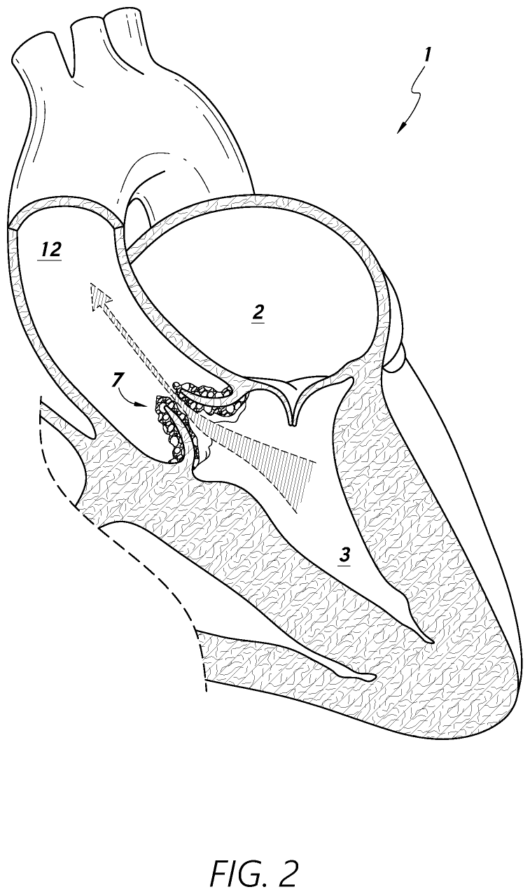

FIG. 2 provides a cross-sectional view of the left ventricle 3 and left atrium 2 of an example heart 1. The diagram of FIG. 2 shows the aortic valve 7, wherein calcification at the aortic valve 7 may cause a narrowed opening for blood flow from the left ventricle 3 to the aorta 12. While some embodiments and/or example may be described herein with respect to the left ventricle 3, aortic valve 7, and/or aorta 12, such embodiments and/or examples may additionally or alternatively be applicable to other portions of the heart 1, including the mitral valve, tricuspid valve, right ventricle, left atrium 2, and/or right atrium, among other valves and/or chambers of the heart 1.

The valve 7 may be configured to at least partially open to allow blood flow through the valve 7. In the example of the aortic valve 7, blood may flow in the upward direction of FIG. 2, from the left ventricle 3 to the aorta 12. If the opening of the leaflets of the valve 7 are relatively close together to create a relatively small opening, the flow velocity through the valve 7 may increase.

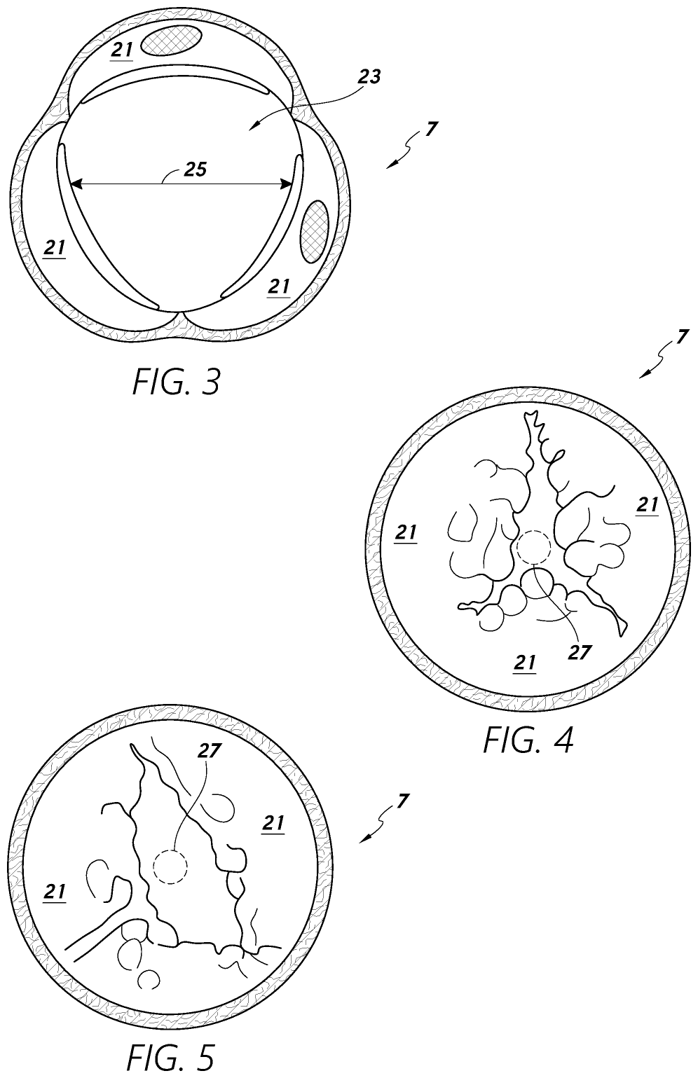

FIGS. 3-5 illustrate overhead views (e.g., from the aorta 12) of example valves 7 (e.g., aortic, mitral, and/or tricuspid valves). An example valve (e.g., a tricuspid valve) can include three leaflets 21. However, valves of the heart 1 may have varying numbers of leaflets 21, for example two or three leaflets 21. Some embodiments described herein may be applicable to valves having any number of leaflets 21.

With respect to a healthy heart valve, the valve leaflets 21 may extend inward from the valve annulus and come together in the flow orifice to permit flow in the outflow direction (e.g., the upward direction in FIG. 2) and prevent backflow or regurgitation toward the inflow direction (e.g., the downward direction in FIG. 2).

The valve leaflets 21 may at least partially open and close during heartbeats to allow blood to flow through the valve 7. FIG. 3 illustrates a healthy valve 7 in an open state, in which the valve 7 creates an opening 23 having a first diameter 25.

However, various conditions may affect the ability of the valve 7 to open and/or close. Heart valve disease represents a condition in which one or more of the valves of the heart fails to function properly. Diseased heart valves may be categorized as stenotic, wherein the valve does not open sufficiently to allow adequate forward flow of blood through the valve, and/or incompetent, wherein the valve does not close completely, causing excessive backward flow of blood through the valve when the valve is in a closed state. In certain conditions, valve disease can be severely debilitating and even fatal if left insufficiently treated.

FIGS. 4 and 5 illustrate example heart valves 7 experiencing calcium buildup and/or accumulated deposits of calcium (i.e., calcification). FIG. 4 illustrates an example valve 7 having three leaflets 21 in an open state while experiencing calcification and FIG. 5 illustrates an example valve 7 having two leaflets 21 in an open state while experiencing calcification.

As shown in FIGS. 4 and 5, the valve 7 may be able to create an opening (in the open state) having a reduced diameter relative the first diameter 25 of the healthy valve 7 shown in FIG. 3. As a result, the valve 7 may provide reduced space for blood to flow through the valve 7 and/or for medical instruments to be delivered through the valve. However, a calcified valve 7 may generally form at least a partial opening at or near a central point 27 of the valve 7. Thus, it may be advantageous to position medical instruments above a central portion of the valve

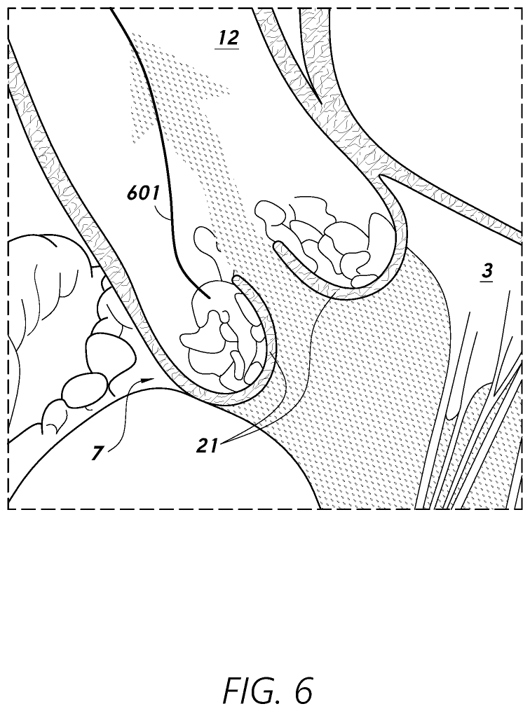

FIG. 6 illustrates a delivery process involving delivering a guidewire 601 and/or similar device to a calcified valve 7 of a heart. A guidewire 601 and/or similar device may be utilized in various treatments at the valve 7 and/or surrounding tissue. In such treatments, a surgeon may navigate the guidewire 601 through the aorta 12 and through the aortic valve 7.

Various factors can present difficulties in navigating the guidewire 601 through the aortic and/or other valve 7. For example, a natural curvature of the aorta 12 can require the surgeon to bend the guidewire 601 to situate an end portion of the guidewire 601 over a central portion of the valve 7 to pass the guidewire 601 through the opening of the valve 7. Moreover, blood flow through the valve 7 can be directed opposite to the direction the guidewire 601 is passed through the valve 7. For example, the guidewire 601 may be passed from the aorta 12 through the aortic valve 7 and into the left ventricle 3 while natural blood flow is from the left ventricle 3 through the aortic valve 7 and into the aorta 12, as indicated by the upward arrow in FIG. 6. This opposing blood flow can cause forces on the guidewire 601 to push the guidewire 601 away from the opening of the valve 7 and/or towards the leaflets 21 and/or surrounding tissue of the valve 7.

Calcification at the valve 7 can also greatly increase the difficulty of navigating the guidewire 601 through the valve 7 due at least in part to narrowing of the opening of the valve 7 caused by calcification. Narrowing of the opening of the valve 7 can limit the space available to surgeons for passing the guidewire 601 through the valve 7. Moreover, a narrowed opening can cause increased blood flow velocity through the aortic valve 7 as the blood has a reduced surface area to travel through. This increased blood flow velocity can further oppose delivery of the guidewire 601 through the valve 7. Moreover, the guidewire 601 can be pushed toward calcified areas of the valve 7 and/or can cause calcified fragments to be displaced from the valve 7 and/or to enter the blood stream, which can result in various issues for the patient.

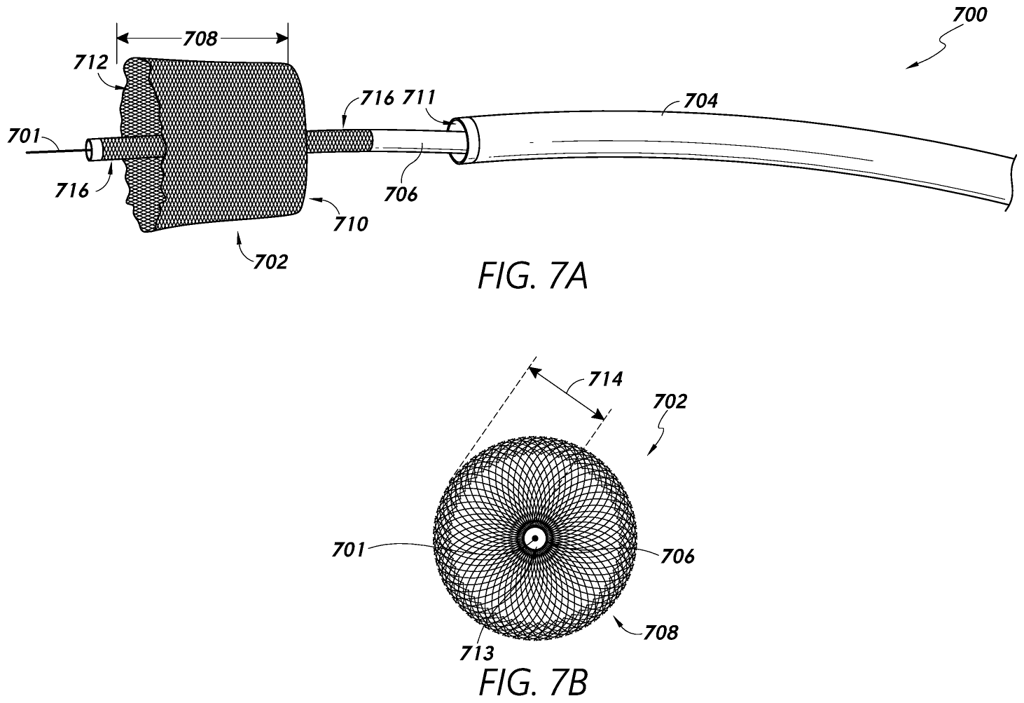

FIGS. 7A and 7B illustrate an example delivery system 700 for delivering a positioning device 702 and/or guidewire 701 into a patient's body for treatment of a heart valve, in accordance with one or more embodiments of the present disclosure. A delivery device 704, which can include a catheter (e.g., a steerable catheter) and/or a sheath (e.g., a steerable sheath) may be configured for navigation through one or more blood flow pathways (e.g., an aorta) and/or chambers (e.g., a left ventricle) of a heart. For example, the delivery device 704 may be delivered through the aorta to the aortic side of the aortic valve. The blood flow pathway may have a generally circular and/or tubular form with tissue walls surrounding a generally tubular hollow interior through which blood may flow. The delivery device 704 may be configured to fit within the blood flow pathway. Moreover, upon removal from the delivery device 704, the positioning device 702 may be configured to expand in multiple directions and/or to conform to a shape of the blood flow pathway. For example, the positioning device 702 may be shape-set to a width and/or diameter slightly larger than a diameter of the blood flow pathway and/or the positioning device 702 may be configured to naturally expand to press against the tissue walls of the blood flow pathway. In some embodiments, the positioning device 702 may be shape-set to a width and/or diameter slightly smaller than the diameter of the blood flow pathway and/or may be configured to loosely fit within the blood flow pathway. The delivery device 704 may comprise a lumen configured to receive the positioning device 702, guidewire 701, and/or an inner catheter 706.

In some embodiments, the positioning device 702 may be configured to at least partially expand within a blood flow pathway (e.g., the aorta) in response to exiting the delivery device 704. For example, the positioning device 702 may be configured to assume a collapsed form while within the delivery device 704 and/or to expand when removed from the delivery device 704.

The positioning device 702 may have any of a variety of forms and/or may be configured to be at least partially crimped onto an exterior surface of the inner catheter 706. The inner catheter 706 may be configured to provide support for the positioning device 702 and/or movement of the inner catheter 706 may be configured to cause corresponding movement of the positioning device 702. For example, extending the inner catheter 706 out of the delivery device 704 may cause the positioning device 702 to extend out of the delivery device 704 and/or pulling the inner catheter 706 back into the delivery device 704 may cause the positioning device 702 to be pulled into the delivery device 704. In some embodiments, the positioning device 702 may be configured to naturally expand when removed from the delivery device 704 and/or to naturally collapse when pulled against the delivery device 704, thereby allowing the positioning device 702 to re-enter the lumen of the delivery device 704.

At least a portion of the positioning device 702 may be configured to crimp and/or attach to an outer/exterior surface of the inner catheter 706. For example, the positioning device 702 may comprise one or more crimped portions 716 configured to form a secure connection to the inner catheter 706 such that the positioning device 702 and/or inner catheter 706 may provide support for each other. The positioning device 702 may comprise an expandable base portion 710 extending from and/or connected to the one or more crimped portions 716. The expandable base portion 710 may be configured to compress in width and/or diameter to fit within the delivery device 704 and/or may be configured to expand in width and/or diameter upon removal from the delivery device 704. In some embodiments, the expandable base portion 710 may have a generally flat profile and/or may be configured to form a generally flat bottom/side of a generally cylindrical shape of the positioning device 702. For example, the positioning device 702 may comprise one or more side portions 708 configured to extend a first length from the expandable base portion 710 to an opening 712 of the positioning device 702. The one or more side portions 708 may have a generally circular and/or tubular form to conform to and/or approximate a shape of the blood flow pathway. In some embodiments, the one or more side portions 708 may be configured to extend in generally flat lines along the tissue walls of the blood flow pathway to prevent tilting of the positioning device 702, inner catheter 706, and/or guidewire 701. For example, the one or more side portions 708 may be configured to extend from the expandable base portion 710 a first length to the opening 712 and/or one or more end portions of the positioning device 702. In some embodiments, the generally cylindrical shape of the positioning device 702 and/or one or more side portions 708 may cause the positioning device 702 and/or one or more side portions 708 to contact and/or press against multiple tissue wall portions of the blood flow pathway to provide support to the positioning device 702, inner catheter 706, and/or guidewire 701.

In some embodiments, the positioning device 702 may be at least partially composed of braided and/or interwoven cords and/or other materials. For example, the positioning device 702 may be at least partially composed of braided shape-memory alloys (e.g., Nitinol). A braided and/or interwoven structure may provide several potential advantages. For example, the positioning device 702 may be configured to allow blood flow through gaps in the material of the positioning device 702 to prevent the positioning device 702 from restricting blood flow in the blood flow pathway.

FIG. 7B provides a frontal view of the positioning device 702, guidewire 701, and inner catheter 706. In some embodiments, the positioning device 702 may have a generally circular form to approximate the forms of various blood flow pathways (e.g., the aorta). The positioning device 702 may be configured to expand outwardly from the guidewire 701 and/or inner catheter 706 in some or all directions such that the guidewire 701 and/or inner catheter 706 may be situated at a center point and/or portion of the positioning device 702. The inner catheter 706 may comprise an inner lumen 713 configured to receive the guidewire 701.

The positioning device 702 may have a generally hollow interior and/or may be configured to form a generally cylindrical shape around the guidewire 701 with a base portion 710 at one end of the positioning device (e.g., similar to a cup and/or basket having a base). In some instances, the positioning device 702 may comprise one or more side portions 708 extending a first length from the base portion 710 of the positioning device 702 to one or more openings 712 and/or end portions of the positioning device 702. An opening 712 of the positioning device 702 may open into a generally hollow interior of the positioning device 702. The inner catheter 706, crimped portion 716, and/or guidewire 701 may be configured to pass through the hollow interior of the positioning device 702. The one or more side portions 708 may have a generally flat shape and/or may be configured to extend along a portion of a blood flow pathway and/or may be configured to prevent the positioning device 702 and/or guidewire 701 from tilting within the blood flow pathway. The positioning device 702 may be configured to form a gap between the guidewire 701 and/or inner catheter 706 and the side portion(s) 708 of the positioning device 702. For example, along the side portion(s) 708, the positioning device 702 may be configured to form a generally circular shape having a first radius 714 of empty space between the guidewire/inner catheter 701/706 and the wall portion(s) 708.

The inner catheter 706, positioning device 702, and/or guidewire 701 may be configured to extend from a distal end of the delivery catheter 704. The delivery catheter 704 may comprise an inner lumen 711 configured to receive (e.g., slidingly receive) the inner sheath 706, positioning device 702, and/or guidewire. Moreover, the guidewire 701 may be configured to extend from a distal end of the inner catheter 706. The inner sheath 706 may comprise an inner lumen configured to receive (e.g., slidingly receive) the guidewire 701 and/or other devices.

The positioning device 702 may comprise an opening 712 into a generally hollow portion of the positioning device 702. In some embodiments, the opening 712 may be positioned across from the base portion 710 of the positioning device 702. In other words, the positioning device 702 may be configured to form a cup and/or basket shape, in which the base portion 710 forms a generally flat bottom and the side portions 708 form one or more walls around the generally hollow portion. The positioning device 702 may be configured to be situated such that the opening 712 is positioned at or near a heart valve and/or an opening of a heart valve. In this way, fragments (e.g., calcified fragments) displaced at or near the heart valve (e.g., by the guidewire 701) may enter the opening 712 and/or the positioning device may be configured to collect the displaced fragments.

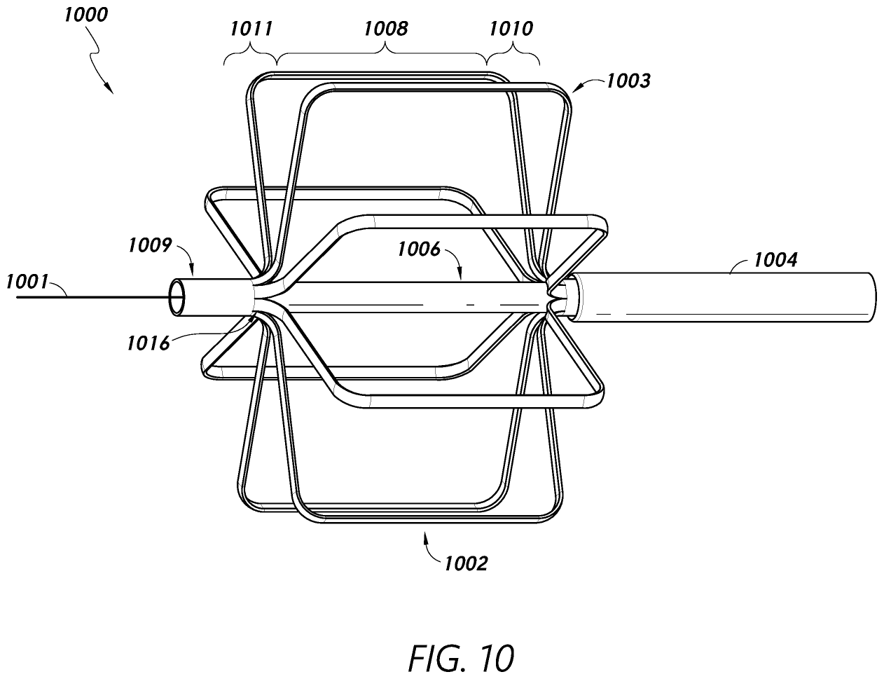

While the positioning device 702 is shown having an opening 712 at one portion of the positioning device 702, the side portion(s) 708 may be configured to connect to the crimped portion at or near the opening 712 to provide support at multiple portions of the side portion(s) 708 (see, e.g., the distal expandable portion 1011 of FIG. 10). For example, the opening 712 may be at least partially covered by one or more cords, wires, and/or braided portions which may be configured to extend across the opening 712 from the side portion(s) 708 to the crimped portion 716. The side portion(s) 708 may be configured to extend into and/or connect to the crimped portion 716 at or near the opening 712 and/or separate connecting elements (e.g., wires, cords, and/or similar devices) may be attached to the positioning device 702 similar to spokes of a wheel to connect the side portion(s) 708 to the crimped portion 716.

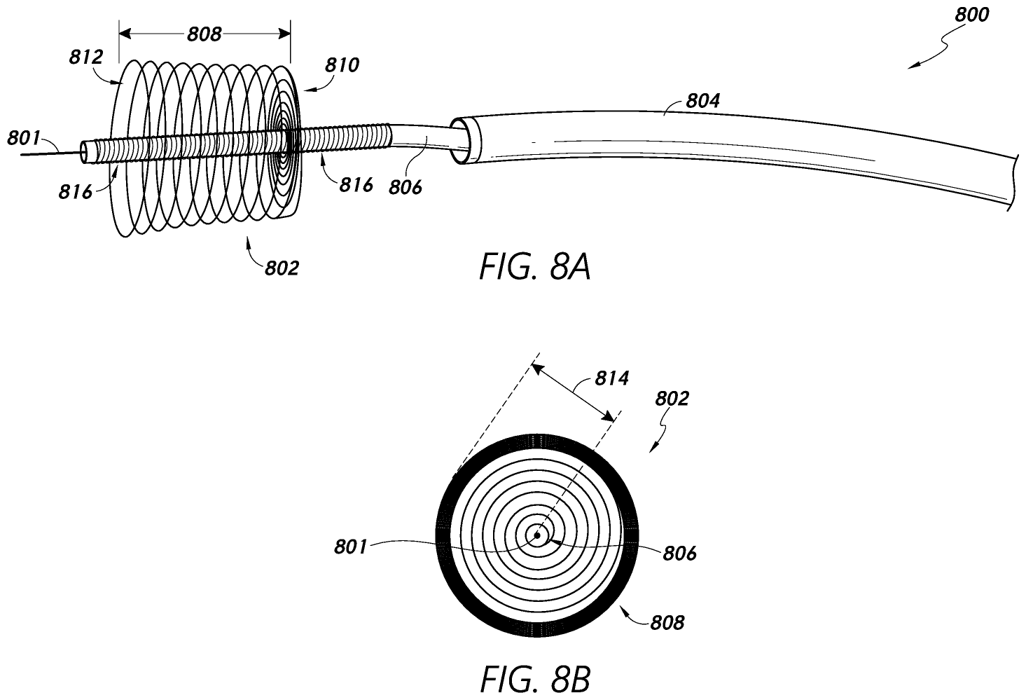

FIGS. 8A and 8B illustrate an example delivery system 800 for delivering a positioning device 802 and/or guidewire 801 into a patient's body for treatment of a heart valve, in accordance with one or more embodiments of the present disclosure. A delivery device 804, which can include a catheter (e.g., a steerable catheter) and/or a sheath (e.g., a steerable sheath) may be configured for navigation through one or more blood flow pathways and/or chambers of a heart. For example, the delivery device 804 may be delivered through the aorta to the aortic side of the aortic valve. The blood flow pathway may have a generally circular and/or tubular form with tissue walls surrounding a generally tubular hollow interior through which blood may flow. The delivery device 804 may be configured to fit within the blood flow pathway. Moreover, upon removal from the delivery device 804, the positioning device 802 may be configured to expand in multiple directions and/or to conform to a shape of the blood flow pathway. For example, the positioning device 802 may be shape-set to a width and/or diameter slightly larger than a diameter of the blood flow pathway and/or the positioning device 802 may be configured to naturally expand to press against the tissue walls of the blood flow pathway. In some embodiments, the positioning device 802 may be shape-set to a width and/or diameter slightly smaller than the diameter of the blood flow pathway and/or may be configured to loosely fit within the blood flow pathway. The delivery device 804 may comprise a lumen configured to receive the positioning device 802, guidewire 801, and/or an inner catheter 806.

In some embodiments, the positioning device 802 may be configured to at least partially expand within a blood flow pathway (e.g., the aorta) in response to exiting the delivery device 804. For example, the positioning device 802 may be configured to assume a collapsed form while within the delivery device 804 and/or to expand when removed from the delivery device 804.

The positioning device 802 may have any of a variety of forms and/or may be at least partially crimped onto an exterior surface of the inner catheter 806. The inner catheter 806 may be configured to provide support for the positioning device 802 and/or movement of the inner catheter 806 may be configured to cause corresponding movement of the positioning device 802. For example, extending the inner catheter 806 out of the delivery device 804 may cause the positioning device 802 to extend out of the delivery device 804 and/or pulling the inner catheter 806 back into the delivery device 804 may cause the positioning device 802 to be pulled into the delivery device 804. In some embodiments, the positioning device 802 may be configured to naturally expand when removed from the delivery device 804 and/or to naturally collapse when pulled against the delivery device 804, thereby allowing the positioning device 802 to re-enter the lumen of the delivery device 804.

At least a portion of the positioning device 802 may be configured to crimp and/or attach to an outer/exterior surface of the inner catheter 806. For example, the positioning device 802 may comprise one or more crimped portions 816 configured to form a secure connection to the inner catheter 806 such that the positioning device 802 and inner catheter 806 may provide support for each other. The positioning device 802 may comprise an expandable base portion 810 extending from and/or connected to the one or more crimped portions 816. The expandable base portion 810 may be configured to compress in width and/or diameter to fit within the delivery device 804 and/or may be configured to expand in width and/or diameter upon removal from the delivery device 804. In some embodiments, the expandable base portion 810 may have a generally flat profile and/or may be configured to form a generally flat bottom/side of a generally cylindrical shape of the positioning device 802. For example, the positioning device 802 may comprise one or more side portions 808 configured to extend a first length from the expandable base portion 810 to an opening 812 of the positioning device 802. The one or more side portions 808 may have a generally circular and/or tubular form to conform to and/or approximate a shape of the blood flow pathway. In some embodiments, the one or more side portions 808 may be configured to extend in generally flat lines along the tissue walls of the blood flow pathway to prevent tilting of the positioning device 802, inner catheter 806, and/or guidewire 801. For example, the one or more side portions 808 may be configured to extend from the expandable base portion 810 a first length to the opening 812 and/or one or more end portions of the positioning device 802. In some embodiments, the generally cylindrical shape of the positioning device 802 and/or one or more side portions 808 may cause the positioning device 802 and/or one or more side portions 808 to contact and/or press against multiple tissue wall portions of the blood flow pathway to provide support to the positioning device 802, inner catheter 806, and/or guidewire 801.

In some embodiments, the positioning device 802 may be at least partially composed of one or more coiled cords and/or wires. For example, the positioning device 802 may be at least partially composed of shape-memory alloys (e.g., Nitinol) shape-set into a coiled form. A coiled structure may provide several potential advantages. For example, the positioning device 802 may be configured to allow blood flow through gaps in the coils of the positioning device 802 to prevent the positioning device 802 from restricting blood flow in the blood flow pathway. Moreover, the coils may be configured to expand (e.g., separate and/or increase in diameter) and/or compress (e.g., move closer together and/or decrease in diameter) as needed to fit within the delivery catheter 804 and/or to fill space within the blood flow pathway. In some embodiments, the positioning device 802 may have a generally soft tip portion and/or may comprise an atraumatic tip to prevent the end portion of the positioning device 802 from piercing and/or damaging the surrounding tissue.

FIG. 8B provides a frontal view of the positioning device 802, guidewire 801, and inner catheter 806. In some embodiments, the positioning device 802 may have a generally circular form to approximate the forms of various blood flow pathways (e.g., the aorta). The positioning device 802 may be configured to expand outwardly from the guidewire 801 and/or inner catheter 806 in some or all directions such that the guidewire 801 and/or inner catheter 806 may be situated at a center point and/or portion of the positioning device 802.

The positioning device 802 may have a generally hollow interior and/or may be configured to form a cup and/or basket shape around the guidewire 801 and/or may comprise one or more side portions 808 extending a first length from a base portion 810 of the positioning device 802 to one or more openings 812 and/or end portions of the positioning device 802. An opening 812 of the positioning device 802 may open into a generally hollow interior of the positioning device 802. The inner catheter 806, crimped portion 816, and/or guidewire 801 may be configured to pass through the hollow interior of the positioning device 802. The one or more side portions 808 may have a generally flat shape and/or may be configured to extend along a portion of a blood flow pathway and/or may be configured to prevent the positioning device 802 and/or guidewire 801 from tilting within the blood flow pathway. The positioning device 802 may be configured to form a gap between the guidewire 801 and/or inner catheter 806 and the side portion(s) 808 of the positioning device 802. For example, along the side portion(s) 808, the positioning device 802 may be configured to form a generally circular shape having a first radius 814 of empty space between the guidewire/inner catheter 801/806 and the side portion(s) 808.

The one or more side portions 808, crimped portions 816, and/or base portion 810 may be composed of one or more cords coiled about the inner catheter 806 and/or guidewire 801. In some embodiments, the base portion 810 may comprise one or more coils which may increase in diameter (e.g., with each successive coil) to increase a diameter of the positioning device 802 from a minimal diameter (e.g., at the crimped portions 816 around the inner catheter 806) to a maximal diameter (e.g., at the one or more side portions 808). In some embodiments, the one or more cords may maintain the maximal diameter between the base portion 810 and the opening 812 of the positioning device 802.

While the positioning device 802 is shown having an opening 812 at one portion of the positioning device 802, the side portion(s) 808 may be configured to connect to the crimped portion at or near the opening 812 to provide support at multiple portions of the side portion(s) 808 (see, e.g., the distal expandable portion 1011 of FIG. 10). For example, the opening 812 may be at least partially covered by one or more cords, wires, and/or braided portions which may be configured to extend across the opening 812 from the side portion(s) 808 to the crimped portion 816. The side portion(s) 808 may be configured to extend into and/or connect to the crimped portion 816 at or near the opening 812 and/or separate connecting elements (e.g., wires, cords, and/or similar devices) may be attached to the positioning device 802 similar to spokes of a wheel to connect the side portion(s) 808 to the crimped portion 816.

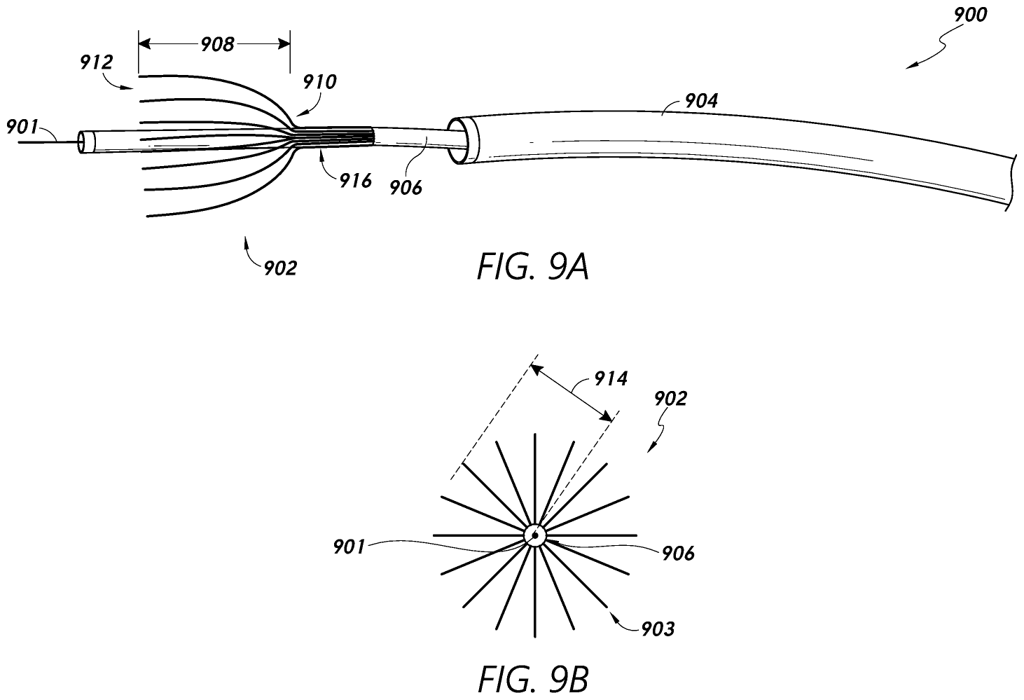

FIGS. 9A and 9B illustrate an example delivery system 900 for delivering a positioning device 902 and/or guidewire 901 into a patient's body for treatment of a heart valve, in accordance with one or more embodiments of the present disclosure. A delivery device 904, which can include a catheter (e.g., a steerable catheter) and/or a sheath (e.g., a steerable sheath) may be configured for navigation through one or more blood flow pathways and/or chambers of a heart. For example, the delivery device 904 may be delivered through the aorta to the aortic side of the aortic valve. The blood flow pathway may have a generally circular and/or tubular form with tissue walls surrounding a generally tubular hollow interior through which blood may flow. The delivery device 904 may be configured to fit within the blood flow pathway. Moreover, upon removal from the delivery device 904, the positioning device 902 may be configured to expand in multiple directions and/or to conform to a shape of the blood flow pathway. For example, the positioning device 902 may be shape-set to a width and/or diameter slightly larger than a diameter of the blood flow pathway and/or the positioning device 902 may be configured to naturally expand to press against the tissue walls of the blood flow pathway. In some embodiments, the positioning device 902 may be shape-set to a width and/or diameter slightly smaller than the diameter of the blood flow pathway and/or may be configured to loosely fit within the blood flow pathway. The delivery device 904 may comprise a lumen configured to receive the positioning device 902, guidewire 901, and/or an inner catheter 906.

In some embodiments, the positioning device 902 may be configured to at least partially expand within a blood flow pathway (e.g., the aorta) in response to exiting the delivery device 904. For example, the positioning device 902 may be configured to assume a collapsed form while within the delivery device 904 and/or to expand when removed from the delivery device 904.

The positioning device 902 may have any of a variety of forms and/or may be at least partially crimped onto an exterior surface of the inner catheter 906. The inner catheter 906 may be configured to provide support for the positioning device 902 and/or movement of the inner catheter 906 may be configured to cause corresponding movement of the positioning device 902. For example, extending the inner catheter 906 out of the delivery device 904 may cause the positioning device 902 to extend out of the delivery device 904 and/or pulling the inner catheter 906 back into the delivery device 904 may cause the positioning device 902 to be pulled into the delivery device 904. In some embodiments, the positioning device 902 may be configured to naturally expand when removed from the delivery device 904 and/or to naturally collapse when pulled against the delivery device 904, thereby allowing the positioning device 902 to re-enter the lumen of the delivery device 904.

At least a portion of the positioning device 902 may be configured to crimp and/or attach to an outer/exterior surface of the inner catheter 906. For example, the positioning device 902 may comprise one or more crimped portions 916 configured to form a secure connection to the inner catheter 906 such that the positioning device 902 and inner catheter 906 may provide support for each other. The positioning device 902 may comprise an expandable base portion 910 extending from and/or connected to the one or more crimped portions 916. The expandable base portion 910 may be configured to compress in width and/or diameter to fit within the delivery device 904 and/or may be configured to expand in width and/or diameter upon removal from the delivery device 904. In some embodiments, the expandable base portion 910 may have a generally flat profile and/or may be configured to form a generally flat bottom/side of a generally cylindrical shape of the positioning device 902. For example, the positioning device 902 may comprise one or more side portions 908 configured to extend a first length from the expandable base portion 910 to an opening 912 of the positioning device 902. The one or more side portions 908 may have a generally circular and/or tubular form to conform to and/or approximate a shape of the blood flow pathway. In some embodiments, the one or more side portions 908 may be configured to extend in generally flat lines along the tissue walls of the blood flow pathway to prevent tilting of the positioning device 902, inner catheter 906, and/or guidewire 901. For example, the one or more side portions 908 may be configured to extend from the expandable base portion 910 a first length to the opening 912 and/or one or more end portions of the positioning device 902. In some embodiments, the generally cylindrical shape of the positioning device 902 and/or one or more side portions 908 may cause the positioning device 902 and/or one or more side portions 908 to contact and/or press against multiple tissue wall portions of the blood flow pathway to provide support to the positioning device 902, inner catheter 906, and/or guidewire 901.

In some embodiments, the positioning device 902 may be at least partially composed of one or more cords 903 and/or wires. For example, the positioning device 902 may be at least partially composed of thin shape-memory alloys (e.g., Nitinol) shape-set into a bent form to expand outwardly from the inner catheter 906 to form a set of cords 903 having a collective diameter greater than the inner catheter 906. The set of cords 903 may be configured to form finger-like elements with gaps between the cords of the set of cords 903. The positioning device 902 may be configured to allow blood flow through gaps in the cords 903 of the positioning device 902 to prevent the positioning device 902 from restricting blood flow in the blood flow pathway. Moreover, the cords 903 may be configured to expand (e.g., bend, separate, and/or increase in diameter) and/or compress (e.g., straighten, move closer together, and/or decrease in diameter) as needed to fit within the delivery catheter 904 and/or to fill space within the blood flow pathway. In some embodiments, the cords 903 of the positioning device 902 may have generally soft tip portions and/or may comprise atraumatic tips to prevent the cords 903 of the positioning device 902 from piercing and/or damaging the surrounding tissue.

FIG. 9B provides a frontal view of the positioning device 902, guidewire 901, and inner catheter 906. In some embodiments, the positioning device 902 may have a generally circular form to approximate the forms of various blood flow pathways (e.g., the aorta). The positioning device 902 may be configured to expand outwardly from the guidewire 901 and/or inner catheter 906 in some or all directions such that the guidewire 901 and/or inner catheter 906 may be situated at a center point and/or portion of the positioning device 902.

The positioning device 902 may have a generally hollow interior and/or may be configured to form a cup and/or basket shape around the guidewire 901 and/or may comprise one or more side portions 908 extending a first length from a base portion 910 of the positioning device 902 to one or more openings 912 and/or end portions of the positioning device 902. An opening 912 of the positioning device 902 may open into a generally hollow interior of the positioning device 902. The inner catheter 906, crimped portion 916, and/or guidewire 901 may be configured to pass through the hollow interior of the positioning device 902. The one or more side portions 908 may have a generally flat shape and/or may be configured to extend along a portion of a blood flow pathway and/or may be configured to prevent the positioning device 902 and/or guidewire 901 from tilting within the blood flow pathway. The positioning device 902 may be configured to form a gap between the guidewire 901 and/or inner catheter 906 and the side portion(s) 908 of the positioning device 902. For example, along the side portion(s) 908, the positioning device 902 may be configured to form a generally circular shape having a first radius 914 of empty space between the guidewire/inner catheter 901/906 and the cord(s) 903.

In some embodiments, the one or more cords 903 may be at least partially flexible and/or may be configured to at least partially bend and/or curve. For example, one or more cords 903 may be configured to curve away from the inner catheter 906 (e.g., at the base portion 910 of the positioning device 902) and/or to extend along the tissue walls of the blood flow pathway (e.g., at the side portions 908 of the positioning device 902). In some embodiments, the one or more cords 903 may bend away from the inner catheter 906 at an approximately 90-degree angle at the base portion 910 and/or may gradually curve and/or bend to extend approximately in parallel with the inner catheter 906 at the side portions 908.

While the positioning device 902 is shown having an opening 912 at one portion of the positioning device 902, the side portion(s) 908 may be configured to connect to the crimped portion at or near the opening 912 to provide support at multiple portions of the side portion(s) 908 (see, e.g., the distal expandable portion 1011 of FIG. 10). For example, the opening 912 may be at least partially covered by one or more cords, wires, and/or braided portions which may be configured to extend across the opening 912 from the side portion(s) 908 to the crimped portion 916. The side portion(s) 908 may be configured to extend into and/or connect to the inner catheter 906 at or near the opening 912 and/or separate connecting elements (e.g., wires, cords, and/or similar devices) may be attached to the positioning device 902 similar to spokes of a wheel to connect the side portion(s) 908 to the inner catheter 906.

FIG. 10 illustrates an example delivery system 1000 for delivering a positioning device 1002 and/or guidewire 1001 into a patient's body for treatment of a heart valve, in accordance with one or more embodiments of the present disclosure. A delivery device 1004, which can include a catheter (e.g., a steerable catheter) and/or a sheath (e.g., a steerable sheath) may be configured for navigation through one or more blood flow pathways and/or chambers of a heart. For example, the delivery device 1004 may be delivered through the aorta to the aortic side of the aortic valve. The blood flow pathway may have a generally circular and/or tubular form with tissue walls surrounding a generally tubular hollow interior through which blood may flow. The delivery device 1004 may be configured to fit within the blood flow pathway. Moreover, upon removal from the delivery device 1004, the positioning device 1002 may be configured to expand in multiple directions and/or to conform to a shape of the blood flow pathway. For example, the positioning device 1002 may be shape-set to a width and/or diameter slightly larger than a diameter of the blood flow pathway and/or the positioning device 1002 may be configured to naturally expand to press against the tissue walls of the blood flow pathway. In some embodiments, the positioning device 1002 may be shape-set to a width and/or diameter slightly smaller than the diameter of the blood flow pathway and/or may be configured to loosely fit within the blood flow pathway. The delivery device 1004 may comprise a lumen configured to receive the positioning device 1002, guidewire 1001, and/or an inner catheter 1006.

In some embodiments, the positioning device 1002 may be configured to at least partially expand within a blood flow pathway (e.g., the aorta) in response to exiting the delivery device 1004. For example, the positioning device 1002 may be configured to assume a collapsed form while within the delivery device 1004 and/or to expand when removed from the delivery device 1004.

The positioning device 1002 may have any of a variety of forms and/or may be at least partially crimped onto an exterior surface of the inner catheter 1006. The inner catheter 1006 may be configured to provide support for the positioning device 1002 and/or movement of the inner catheter 1006 may be configured to cause corresponding movement of the positioning device 1002. For example, extending the inner catheter 1006 out of the delivery device 1004 may cause the positioning device 1002 to extend out of the delivery device 1004 and/or pulling the inner catheter 1006 back into the delivery device 1004 may cause the positioning device 1002 to be pulled into the delivery device 1004. In some embodiments, the positioning device 1002 may be configured to naturally expand when removed from the delivery device 1004 and/or to naturally collapse when pulled against the delivery device 1004, thereby allowing the positioning device 1002 to re-enter the lumen of the delivery device 1004.

At least a portion of the positioning device 1002 may be configured to crimp and/or attach to multiple portions of an outer/exterior surface of the inner catheter 1006. For example, the positioning device 1002 may comprise one or more crimped portions 1016 configured to form a secure connection to the inner catheter 1006 such that the positioning device 1002 and inner catheter 1006 may provide support for each other. The positioning device 1002 may comprise a proximal expandable portion 1010 and/or distal expandable portion 1011 extending from and/or connected to the one or more crimped portions 1016. The proximal expandable portion 1010 and/or distal expandable portion 1011 may be configured to compress in width and/or diameter to fit within the delivery device 1004 and/or may be configured to expand in width and/or diameter upon removal from the delivery device 1004. In some embodiments, the proximal expandable portion 1010 and/or distal expandable portion 1011 may have a generally flat profile and/or may be configured to form a generally flat bottom/top/side of a generally cylindrical shape of the positioning device 1002. For example, the positioning device 1002 may comprise one or more side portions 1008 configured to extend a first length from the proximal expandable portion 1010 to the distal expandable portion 1011. The diameter of the positioning device 1002, in the expanded configuration shown in FIG. 10, may be variable at the proximal expandable portion 1010 and/or distal expandable portion 1011 and/or may be generally constant at the one or more side portions 1008. For example, the diameter of the positioning device 1002 may gradually increase/decrease at the proximal expandable portion 1010 and/or distal expandable portion 1011. By attaching to and/or contacting the inner catheter 1006 at multiple portions (e.g., a proximal portion and a distal portion), the positioning device 1002 may have improved support from the inner catheter 1006.

While the positioning device 1002 is shown having seven distinct cords 1003 distributed evenly about the inner catheter 1006, the positioning device 1002 may comprise any number of cords 1003 and/or the cords 1003 may be distributed in any manner. In some embodiments, one or more cords 1003 may be inter-connected and/or may extend into each other. The cords 1003 may be composed of any suitable material, which can include various shape-memory alloys (e.g., Nitinol). For example, the positioning device 1002 may be formed at least partially from a Nitinol hypotube cut and/or shape-set into the form shown in FIG. 10. The positioning device 1002 may advantageously present a relatively low profile to allow the positioning device 1002 to be compressed and/or crimped onto the inner catheter 1006 and/or into the delivery device 1004.

The one or more side portions 1008 may have a generally circular and/or tubular form about the inner catheter 1006 to conform to and/or approximate a shape of the blood flow pathway. In some embodiments, the one or more side portions 1008 may be configured to extend in generally flat lines along the tissue walls of the blood flow pathway to prevent tilting of the positioning device 1002, inner catheter 1006, and/or guidewire 1001. For example, the one or more side portions 1008 may be configured to extend from the proximal expandable portion 1010 a first length to the distal expandable portion 1011. In some embodiments, the generally cylindrical shape of the positioning device 1002 and/or one or more side portions 1008 may cause the positioning device 1002 and/or one or more side portions 1008 to contact and/or press against multiple tissue wall portions of the blood flow pathway to provide support to the positioning device 1002, inner catheter 1006, and/or guidewire 1001.

In some embodiments, the positioning device 1002 may be at least partially composed of one or more cords 1003 and/or wires. For example, the positioning device 1002 may be at least partially composed of thin shape-memory alloys (e.g., Nitinol) shape-set into a bent form to expand outwardly from the inner catheter 1006 to form a set of cords 1003 having a collective diameter greater than the inner catheter 1006. The set of cords 1003 may be configured to form an enclosure similar to a cage about the inner catheter 1006 with gaps between the cords 1003 of the set of cords 1003. The positioning device 1002 may be configured to allow blood flow through gaps in the cords 1003 of the positioning device 1002 to prevent the positioning device 1002 from restricting blood flow in the blood flow pathway. Moreover, the cords 1003 may be configured to expand (e.g., bend, separate, and/or increase in diameter) and/or compress (e.g., straighten, move closer together, and/or decrease in diameter) as needed to fit within the delivery catheter 1004 and/or to fill space within the blood flow pathway.

The delivery system 1000 may further include a sheath 1009 situated between the inner sheath 1006 and the delivery device 1004. However, the sheath 1009 may not necessarily be included in some embodiments. The positioning device 1002 may be configured to extend between the inner catheter 1006 and the sheath. In some embodiments, the positioning device 1002 may be configured to at least partially slide along the inner catheter 1006 and/or sheath 1009 to form the expanded configuration shown in FIG. 10 and/or collapse to fit into the delivery device 1004.

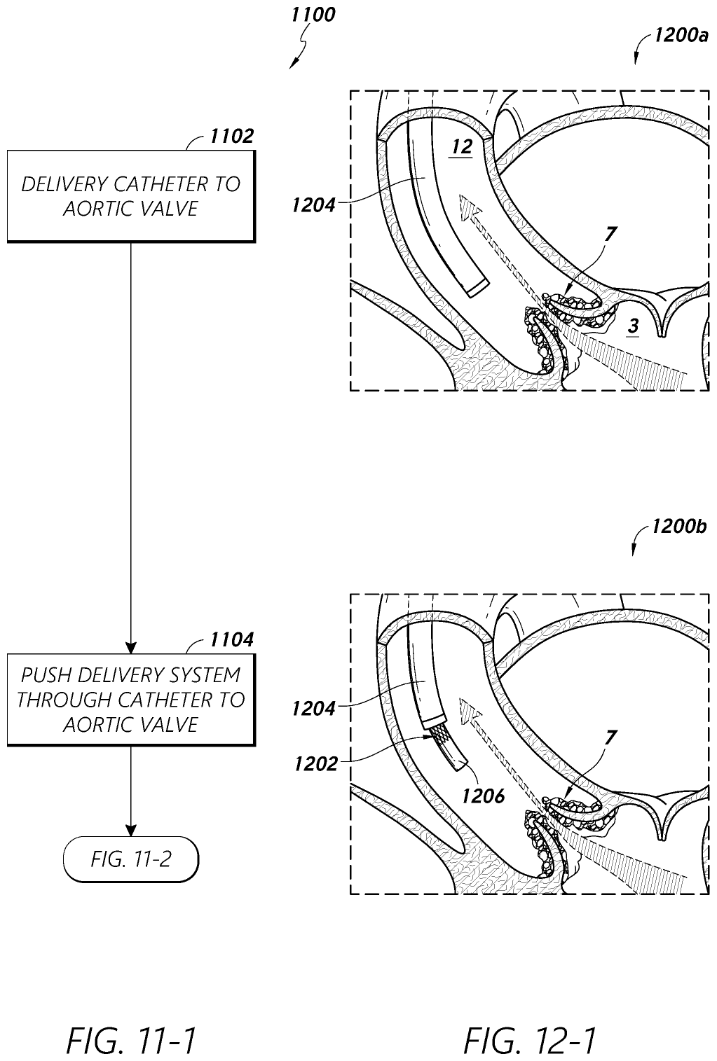

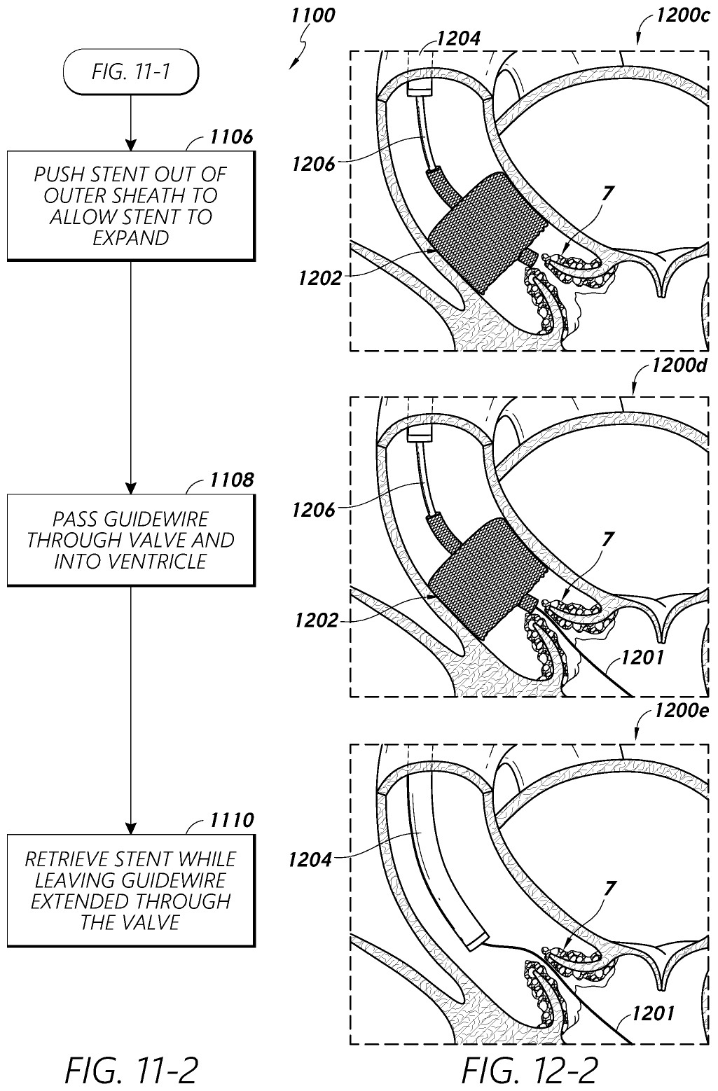

FIGS. 11-1 and 11-2 are flowcharts illustrating steps of a process 1100 for delivering a guidewire and/or similar device through a valve which may be at least partially narrowed due to calcification and/or other factors, in accordance with one or more embodiments of the present disclosure. FIGS. 12-1 and 12-2 provide images relating to steps of the process 1100 of FIGS. 11-1 and 11-2. While FIGS. 12-1 and 12-2 illustrate a positioning device 1202 composed of braided materials, other types and/or forms of positioning devices 1202 may apply to steps of the process 1100 described in FIGS. 11-1 and 11-2.

At step 1102, the process 1100 involves delivering a catheter 1204 through a blood flow pathway (e.g., the aorta 12) to a valve (e.g., the aortic valve 7), as shown in image 1200a of FIG. 12-1. In some embodiments, the catheter 1204 may comprise a steerable catheter configured to at least partially bend to navigate around bends of the blood flow pathway. The catheter 1204 may be configured for delivering one or more implants and/or delivery devices. For example, the catheter 1204 may comprise a lumen configured to receive one or more medical instruments.

While the aortic valve 7 is described with respect to FIGS. 11-1, 11-2, 12-1, and 12-2, steps of the process 1100 may be applicable to delivery through other valves of the heart. The valve 7 may represent an opening for blood flow from the left ventricle 3 to the aorta 12. Blood may naturally flow from the left ventricle 3 to the aorta 12. Thus, delivery of various devices including guidewires from the aorta 12 to the left ventricle 3 may be upstream of the natural blood flow and/or the natural blood flow may oppose the delivery of the various devices from the aorta 12 to the left ventricle 3.

At step 1104, the process 1100 involves extending a delivery system configured for delivery of a guidewire and/or other devices through the catheter 1204 and/or an inner sheath 1206 into close proximity of the valve 7, as shown in image 1200b of FIG. 12-1. In some embodiments, the delivery system may comprise a positioning device 1202 (e.g., a stent and/or similar device) crimped onto an inner sheath 1206 delivered through a lumen of the catheter 1204. The inner sheath 1206 may be configured to provide support to the positioning device 1202.

At step 1106, the process 1100 involves extending the positioning device 1202 until the positioning device 1202 exits the catheter 1204 and/or expands within the blood flow pathway, as shown in image 1200c of FIG. 12-2. The positioning device 1202 may have any of the variety of forms described herein and/or may be configured to extend in multiple and/or all directions around the inner sheath 1206. The positioning device 1202 may be configured to press against tissue walls of the blood flow pathway (e.g., the aorta) to prevent the inner sheath 1206 from drifting away from a central portion of the blood flow pathway. For example, blood flow through the valve 7 may cause pushing force against the inner sheath 1206 and/or positioning device 1202 to press the inner sheath 1206 and/or positioning device 1202 away from an opening of the valve 7. However, the positioning device 1202 may be configured to counteract such forces by pressing against the tissue walls of the blood flow pathway to prevent drifting. In some embodiments, the positioning device 1202 may be at least partially composed of a network of thin materials (e.g., metallic cords) with gaps between the materials to thereby allow blood flow through the positioning device 1202 and/or to prevent blockage in the blood flow pathway.

At step 1108, the process 1100 involves extending a guidewire 1201 through and/or out of the catheter 1204 and/or inner sheath 1206 and/or through the valve 7, as shown in image 1200d of FIG. 12-2. When the guidewire 1201 exits the inner sheath 1206 and/or positioning device 1202, the guidewire 1201 may advantageously be situated approximately over a central portion of the valve 7 due at least in part to the positioning device 1202 pressing the inner sheath 1206 and/or guidewire 1201 toward the central portion of the valve 7.

At step 1110, the process 1100 involves retrieving the positioning device 1202 back into the catheter 1204 while leaving the guidewire 1201 extended through the valve 7, as shown in image 1200e of FIG. 12-2. In some embodiments, retrieving the positioning device 1202 may involve pulling the inner sheath 1206 and/or positioning device 1202 back toward the catheter 1204. The positioning device 1202 may be configured to naturally collapse in response to being pulled backward against the catheter 1204. For example, the catheter 1204 may be configured to press against a bottom surface of the positioning device 1202 and/or the positioning device 1202 may be configured to naturally collapse in response to pressing forces at the bottom surface of the positioning device 1202.

Depending on the embodiment, certain acts, events, or functions of any of the processes or algorithms described herein can be performed in a different sequence, may be added, merged, or left out altogether. Thus, in certain embodiments, not all described acts or events are necessary for the practice of the processes. Moreover, in certain embodiments, acts or events may be performed concurrently, e.g., through multi-threaded processing, interrupt processing, or via multiple processors or processor cores, rather than sequentially.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y and Z,” unless specifically stated otherwise, is understood with the context as used in general to convey that an item, term, element, etc. may be either X, Y or Z. Thus, such conjunctive language is not generally intended to imply that certain embodiments require at least one of X, at least one of Y and at least one of Z to each be present.

It should be appreciated that in the above description of embodiments, various features are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular embodiment herein can be applied to or used with any other embodiment(s). Further, no component, feature, step, or group of components, features, or steps are necessary or indispensable for each embodiment. Thus, it is intended that the scope of the inventions herein disclosed and claimed below should not be limited by the particular embodiments described above, but should be determined only by a fair reading of the claims that follow.

What is claimed is:

1. A system for delivering a guidewire through a heart valve, the system comprising:
a delivery catheter configured to pass through a blood flow pathway of a heart;
an inner catheter having an inner lumen;
a guidewire configured to pass through the inner lumen of the inner catheter; and
a positioning device crimped onto an outer surface of the inner catheter;
wherein:
the delivery catheter is configured to receive the inner catheter, guidewire, and positioning device;
the delivery catheter is configured to prevent expansion of the positioning device; and
the positioning device is configured to expand upon removal from the delivery catheter.

2. The system of claim 1, wherein the positioning device is configured to conform to the blood flow pathway upon removal from the delivery catheter.

3. The system of claim 1, wherein the positioning device is configured to, upon removal from the delivery catheter, position the inner catheter and guidewire above a central portion of the heart valve.

4. The system of claim 1, wherein the positioning device is at least partially composed of braided cords.

5. The system of claim 1, wherein the positioning device has a generally cylindrical shape with generally flat sides configured to extend along the blood flow pathway to prevent tilting of the positioning device.

6. The system of claim 1, wherein the positioning device comprises:
a base portion;
one or more side portions extending from the base portion and around a hollow interior of the positioning device; and
an opening across from the base portion and configured to be positioned near the heart valve to collect fragments displaced from the heart valve.

7. The system of claim 1, wherein the positioning device is configured to expand to a diameter that is less than a diameter of the blood flow pathway to allow the positioning device to move along the blood flow pathway after the positioning device expands.

8. The system of claim 1, wherein the positioning device comprises one or more cords coiled about the inner catheter.

9. The system of claim 8, wherein the one or more cords are configured to increase in diameter at a base portion of the positioning device and maintain a maximal diameter at a side portion of the positioning device.

10. The system of claim 1, wherein the positioning device comprises one or more flexible cords configured to curve away from the inner catheter and extend along the blood flow pathway.

11. The system of claim 1, wherein the positioning device is configured to attach at multiple portions of the outer surface of the inner catheter.

12. A method for delivering a guidewire through a heart valve, the method comprising:
delivering a delivery catheter via a blood flow pathway to a heart valve, the delivery catheter having a first inner lumen configured to receive an inner catheter, a positioning device, and a guidewire;
passing the inner catheter through the first inner lumen of the delivery catheter, the inner catheter having a second inner lumen and configured to carry the positioning device at an exterior surface of the inner catheter;
extending the inner catheter out of a distal end of the delivery catheter to allow the positioning device to expand in multiple directions around the inner catheter; and
passing the guidewire through the second inner lumen of the inner catheter and out of a distal end of the inner catheter.

13. The method of claim 12, wherein the positioning device is configured to expand to position the inner catheter and guidewire above a central portion of the heart valve.

14. The method of claim 12, wherein the positioning device has a generally cylindrical shape with generally flat sides configured to extend along the blood flow pathway to prevent tilting of the positioning device.

15. The method of claim 12, wherein the positioning device comprises:
a base portion;
one or more side portions extending from the base portion and around a hollow interior of the positioning device; and
an opening across from the base portion and configured to be positioned near the heart valve to collect fragments displaced from the heart valve.

16. The method of claim 12, wherein the positioning device comprises one or more cords coiled about the inner catheter.

17. A device for positioning a guidewire at an opening of a heart valve, the device comprising:
a crimped portion configured to crimp to an inner catheter;
an expandable base portion configured to compress to fit within a delivery catheter and expand within a blood flow pathway upon removal from the delivery catheter; and
one or more side portions configured to extend along tissue walls of the blood flow pathway to prevent tilting of the inner catheter.

18. The device of claim 17, wherein the expandable base portion is at least partially composed of braided cords.

19. The device of claim 17, wherein the expandable base portion comprises one or more cords coiled about the inner catheter.

20. The device of claim 17, wherein the expandable base portion and one or more side portions comprise one or more flexible cords configured to curve away from the inner catheter and extend along the blood flow pathway.
